(19) United States
(12) Patent Application Publication
(10) Pub. No.: US 2022/0220149 A1
(43) Pub. Date: Jul. 14, 2022

(54) SOLID STATE FORMS OF SAGE-217 AND PROCESSES FOR PREPARATION THEREOF

(71) Applicant: TEVA CZECH INDUSTRIES S.R.O, Opava-Komarov (CZ)

(72) Inventors: Polina Lapido, Rishon Lezion (IL); Ofir Shaul, Hod Hasharon (IL); Firas Masri, Nazareth (IL)

(21) Appl. No.: 17/610,192

(22) PCT Filed: May 14, 2020
(86) PCT No.: PCT/US20/32788

§ 371 Date: Nov. 10, 2021

Related U.S. Application Data
(60) Provisional application No. 62/853,229, filed on May 28, 2019.

(51) Int. Cl. C07J 43/00

(52) U.S. Cl. CPC C07J 43/003; C07B 2200/13

(57) ABSTRACT

The present disclosure encompasses solid state forms of SAGE-217, in embodiments SAGE-217:Oxalic acid Co-crystal, processes for preparation thereof, and pharmaceutical compositions thereof.

### FIELD OF THE DISCLOSURE

The present disclosure encompasses a solid-state form of SAGE-217, in embodiments SAGE-217: Oxalic acid Co-crystal, processes for preparation thereof, and pharmaceutical compositions thereof.

### BACKGROUND OF THE DISCLOSURE

SAGE-217, known as Zuranolone, -(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile, has the following chemical structure:

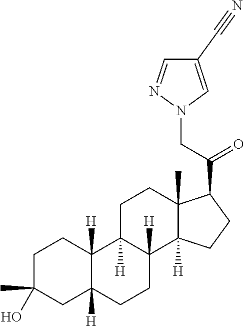

SAGE-217 is a potent GABAA receptor agonist. It is also a potent GABAA receptor modulator at both synaptic and extrasynaptic receptor subtypes, with excellent oral DMPK properties. SAGE-217 is developed for the treatment of CNS related disorders such as postpartum depression (PPD), major depressive disorder (MDD), and essential tremor (ET). The compound is described in U.S. Pat. No. 9,512,165. Solid state forms of SAGE-217 are described in PCT Patent Application Publication No. WO 2018/039378.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis—“TGA”, or differential scanning calorimetry—“DSC”), X-ray diffraction (XRD) pattern, infrared absorption fingerprint, and solid state (¹³C) NMR spectrum. One or more of these techniques may be used to distinguish different polymorphic forms of a compound.

Different salts or co-crystals and solid state forms (including solvated forms) of an active pharmaceutical ingredient may possess different properties. Such variations in the properties of different salts or co-crystals and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts or co-crystals and solid state forms may also offer improvements to the final dosage form, for instance, if they serve to improve bioavailability. Different salts or co-crystals and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms, salts, co-crystals and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms, salts and co-crystals of a pharmaceutically useful compound can also provide an opportunity to improve the performance characteristics of a pharmaceutical product. It enlarges the repertoire of materials that a formulation scientist has available for formulation optimization, for example by providing a product with different properties, such as a different crystal habit, higher crystallinity, or polymorphic stability, which may offer better processing or handling characteristics, improved dissolution profile, or improved shelf-life (chemical/physical stability). Additional solid state forms (including solvated forms), salts and co-crystals of SAGE-217 remain desirable.

### SUMMARY

The present disclosure provides crystalline SAGE-217:oxalic acid, processes for preparation thereof, and pharmaceutical compositions thereof.

The present disclosure provides crystalline SAGE-217:oxalic acid for use in the preparation of pharmaceutical compositions and/or formulations for use in medicine, such as for the treatment of patients having CNS related disorders such as postpartum depression (PPD), major depressive disorder (MDD), and/or essential tremor (ET).

The present disclosure also encompasses the use of crystalline SAGE-217:oxalic acid of the present disclosure for the preparation of pharmaceutical compositions and/or formulations.

In another aspect, the present disclosure provides pharmaceutical compositions including crystalline SAGE-217:oxalic acid according to the present disclosure.

The present disclosure further provides the use of crystalline SAGE-217:oxalic acid as defined in any of the present disclosure for the preparation of other solid state forms of SAGE-217 including co-crystals, hydrates, solvates and anhydrous forms thereof.

In yet another embodiment, the present disclosure encompasses pharmaceutical formulations including any one or a combination of the described crystalline SAGE-217:oxalic acid or pharmaceutical compositions including the described crystalline SAGE-217:oxalic acid and at least one pharmaceutically acceptable excipient.

The present disclosure includes processes for preparing the above mentioned pharmaceutical compositions. The processes include combining any one or a combination of crystalline SAGE-217:oxalic acid with at least one pharmaceutically acceptable excipient.

The crystalline SAGE-217:oxalic acid as defined herein and the pharmaceutical compositions or formulations of the crystalline SAGE-217:oxalic acid may be used as medicaments for the treatment of patients with CNS related disorders such as postpartum depression (PPD), major depressive disorder (MDD), or essential tremor (ET).

The present disclosure also provides methods of treating patients with depression including postpartum depression and major depression, by administering a therapeutically effective amount of any one or a combination of the crystalline SAGE-217:oxalic acid of the present disclosure, or at least one of the above pharmaceutical compositions or formulations, to a subject suffering from depression, or otherwise in need of the treatment.

The present disclosure also provides the uses of crystalline SAGE-217:oxalic acid of the present disclosure, or at least one of the above pharmaceutical compositions or formulations, for the manufacture of medicaments for treating e.g., depression.

### BRIEF DESCRIPTION OF THE FIGURES

FIG. 1 shows a characteristic X-ray powder diffraction pattern (XRPD) of SAGE-217 Form A, as described in PCT Patent Application Publication No. WO 2018/039378.

FIG. 2 shows a characteristic XRPD of crystalline SAGE-217:oxalic acid Form OCC1.

### DETAILED DESCRIPTION

The present disclosure encompasses a solid state form of SAGE-217:oxalic acid, including crystalline polymorph of SAGE-217:oxalic acid, process for preparation thereof, uses and pharmaceutical compositions thereof.

The present disclosure encompasses a solid state form of SAGE-217:oxalic acid Form OCC1, processes for preparation thereof, and pharmaceutical compositions thereof.

Solid state properties of SAGE-217:oxalic acid and crystalline polymorphs thereof can be influenced by controlling the conditions under which SAGE-217:oxalic acid and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of SAGE-217:oxalic acid described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of SAGE-217:oxalic acid. In some embodiments of the disclosure, the described crystalline polymorph of SAGE-217:oxalic acid may contain from about 1% to about 20% (w/w), from about 5% to about 20% (w/w), or from about 5% to about 10% (w/w) of one or more other crystalline polymorph of the same SAGE-217:oxalic acid. In some embodiments of the disclosure, the described crystalline polymorph of SAGE-217:oxalic acid may contain from about 1% to about 20% (w/w), from about 5% to about 20% (w/w), or from about 5% to about 10% (w/w) of one or more other crystalline polymorphs of the same SAGE-217:oxalic acid.

Depending on other crystalline polymorphs with which a comparison is made, the crystalline SAGE-217:oxalic acid of the present disclosure has advantageous properties selected from at least one of the following: chemical purity, flowability, solubility, dissolution rate, morphology or crystal habit, stability (such as chemical stability as well as thermal and mechanical stability with respect to polymorphic conversion), stability towards dehydration and/or storage stability, low content of residual solvent, a lower degree of hygroscopicity, flowability, and advantageous processing and handling characteristics such as compressibility, and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of SAGE-217:oxalic acid referred to herein as being characterized by graphical data “as depicted in” or “as substantially depicted in” a Figure will thus be understood to include any crystal forms of SAGE-217:oxalic acid characterized with the graphical data having such small variations, as are well known to the skilled person, in comparison with the Figure.

As used herein, and unless stated otherwise, the term “anhydrous” in relation to crystalline forms of SAGE-217:oxalic acid, relates to a crystalline form of SAGE-217:oxalic acid which does not include any crystalline water (or other solvents) in a defined, stoichiometric amount within the crystal. Moreover, an “anhydrous” form would typically not contain more than 1% (w/w), of either water or organic solvents as measured, for example, by TGA. In embodiments, the crystalline form of SAGE-217:oxalic acid contains less than 1% (w/w) or less than 0.8 (w/w) of water or organic solvents as measured by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystals” as used herein is defined as a crystalline material including two or more molecules in the same crystalline lattice and associated by non-ionic and non-covalent bonds. In some embodiments, the co-crystal includes two molecules which are in a natural state. In an embodiment the molar ratio between the active pharmaceutical ingredient (SAGE-217) and the coformer (oxalic acid) is between 2:1.5 and 1.5:1, preferably between 2:1.25 and 1.25:1, in other embodiments about 2:1.

As used herein, the term “isolated” in reference to crystalline polymorph of SAGE-217:oxalic acid of the present disclosure corresponds to a crystalline polymorph of SAGE-217 that is physically separated from the reaction mixture in which it is formed.

As used herein, unless stated otherwise, the XRPD measurements are taken using copper Kα radiation wavelength 1.54187 Å. XRPD peaks reported herein are measured using CuKα radiation, λ=1.54187 Å, typically at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Generally, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in embodiments about 16 hours.

As used herein and unless indicated otherwise, the term “ambient conditions” refer to atmospheric pressure and a temperature of 22-24° C.

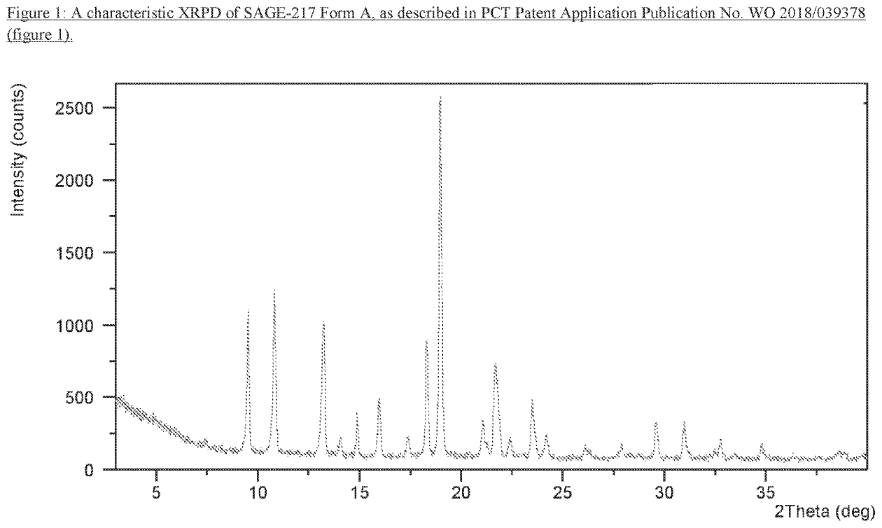

As used herein, the term “SAGE Form A” is Form A which is described in PCT Publication No. WO 2018/039378. For example, as defined in WO2018/039378, Form A has an XRPD pattern with characteristic peaks at: 9.5, 10.8, 13.2, 18.9 and 21.6 degrees 2-theta, or an XRPD pattern with characteristic peaks at: 9.5, 10.8, 13.2, 14.9, 16.0, 18.3, 18.9, 21.1, 21.6 and 23.5 degrees 2-theta. Form A may also be defined with reference to the XRPD in FIG. 1 herein.

As used herein, crystalline SAGE-217:oxalic acid is a distinct molecular species. In one embodiment crystalline SAGE-217:oxalic acid may be a co-crystal of SAGE-217 and oxalic acid.

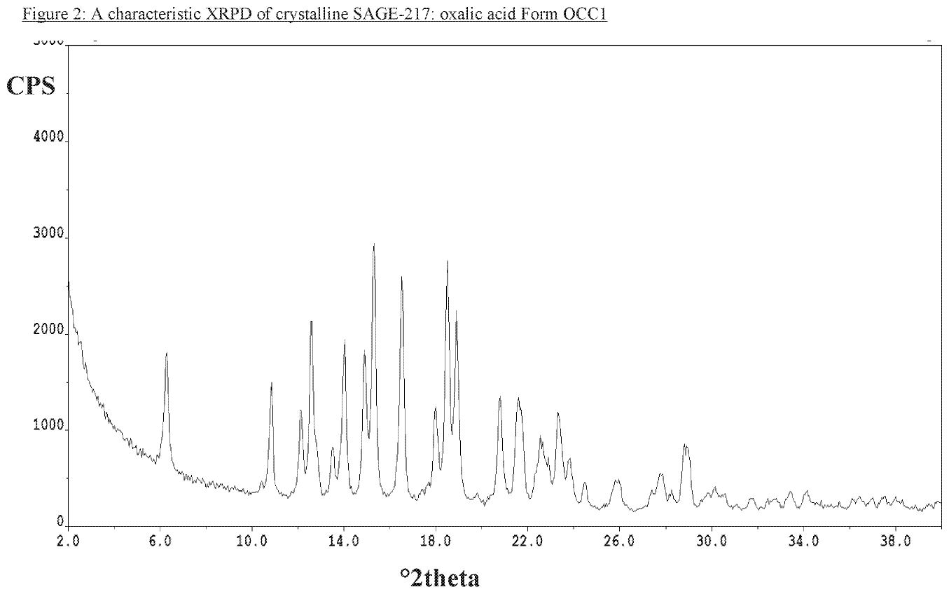

The present disclosure includes a crystalline SAGE-217:oxalic acid, designated Form OCC1. The crystalline Form OCC1 of SAGE-217:oxalic acid may be characterized by data selected from one or more of the following: an X-ray powder diffraction pattern substantially as depicted in FIG. 2; an X-ray powder diffraction pattern having peaks at 6.2, 12.5, 14.0, 15.2 and 18.9 degrees 2-theta±0.2 degrees 2-theta; and combinations of these data.

Crystalline SAGE-217:oxalic acid Form OCC1 may be further characterized by an X-ray powder diffraction pattern having peaks at 6.2, 12.5, 14.0, 15.2 and 18.9 degrees 2-theta±0.2 degrees 2-theta, and also having any one, two, three, four or five additional peaks selected from one or more of 10.8, 16.5, 17.9, 18.5 and 20.7 degrees 2-theta±0.2 degrees 2-theta.

In embodiments of the present disclosure, crystalline Form OCC1 of SAGE-217: oxalic acid is isolated.

In one embodiment crystalline SAGE-217:oxalic acid Form OCC1 may be a co-crystal.

Crystalline Form OCC1 of SAGE-217:oxalic acid may be an anhydrous form.

In another embodiment of the present disclosure, crystalline SAGE-217:oxalic acid Form OCC1 is polymorphically pure.

Crystalline SAGE-217:oxalic acid Form OCC1 may be characterized by each of the above characteristics alone or by all possible combinations, e.g., an XRPD pattern having peaks at 6.2, 10.8, 12.5, 14.0, 15.2, 16.5, 17.9, 18.5 18.9 and 20.7 degrees 2-theta±0.2 degrees 2-theta; an XRPD pattern as depicted in FIG. 2 and combinations thereof.

As described above, depending on which other solid state it is compared with, crystalline SAGE-217:oxalic acid Form OCC1 according to the present disclosure may have advantageous properties as described above, for example stability and improved morphology.

In some embodiments the disclosure relates to processes for preparation of crystalline SAGE-217:oxalic acid Form OCC1.

In one aspect, the disclosure relates to a process for the preparation of crystalline SAGE-217:oxalic acid Form OCC1, wherein the process comprises:
(a) preparing a mixture comprising SAGE-217 and oxalic acid in one or more organic solvents;
(b) isolating crystalline SAGE-217:oxalic acid from the mixture; and optionally
(c) drying.

The organic solvent in step (a) may be a solvent or a mixture of solvents in which the SAGE-217 and oxalic acid are soluble, either at room temperature or with heating. The one or more organic solvents can alternatively include a mixture of a solvent and an antisolvent.

In one embodiment of the process for preparing crystalline SAGE-217:oxalic acid, step (a) comprises providing a mixture containing SAGE-217 and oxalic acid in a solution with at least one solvent, optionally heating to obtain a solution, and optionally filtering the solution to remove insoluble matter. Step (b) may comprise cooling, or removing the solvent by evaporation, preferably at reduced pressure. Preferably, the solvent removal is carried out by evaporation at reduced pressure, wherein the evaporation may be conducted rapidly to dryness in order to form the solid product.

In embodiments, the process for preparing crystalline SAGE-217:oxalic acid Form OCC1 comprises: (a) dissolving SAGE-217 and oxalic acid in one or more organic solvents having a low boiling point under elevated temperature to form a solution, and optionally filtering the solution; (b) isolating crystalline SAGE-217:oxalic acid Form OCC1 by evaporation; and (c) drying.

In embodiments the solution in step (a) has a mol ratio of SAGE-217 to oxalic acid of about 1:1 to about 1:3, about 1:1 to about 1:2, about 1:1 to about 1:1.5, or about 1:1 to about 1:4, or about 1:1 to about 1:0.5, or about 1:0.8 to about 1:0.5, or about 1:0.6 to about 1:0.5, or about 1:0.5. In other embodiments, the solution in step (a) has a mol ratio of SAGE-217 to oxalic acid of about 1:0.8 to about 1:0.5, or about 1:0.6 to about 1:0.5, or about 1:0.5.

In embodiments, the solvent in step (a) comprises one or more solvents having a boiling point of about 80° C. or less, about 70° C. or less, or from about 30-80° C., or from about 40-70° C., or from about 50-70° C. In embodiments, the solvent in step (a) is selected from methanol, acetone and THF, preferably methanol.

In embodiments, the solution in stage (a) the solvent is present at an amount of about 15 to 60 vol, about 15 to 50 vol, about 18 to 45 vol, about 18 to 30 vol, about 18 to 25 vol, and preferably about 24 vol in relation to SAGE-217.

In embodiments the solution in step (a) is preferably heated, preferably to a temperature of about 40 to about 80° C., about 45 to about 70° C., about 45 to about 65° C., and in another embodiments to about 48 to about 65° C., optionally with stirring. Optionally the solution in step (a) is filtered prior to step (b), for example to remove insoluble material before product isolation.

In embodiments, crystalline SAGE-217:oxalic acid Form OCC1 may be isolated in step (b) by cooling or preferably by removal of solvent from the mixture in step (a), for example by evaporation. Evaporation may be performed at a temperature of about 30 to about 60° C., about 40 to 60° C., and in some embodiments about 50° C., preferably under vacuum, particularly wherein a reduced pressure of about 1 to about 100 mbar, about 10 to 50 mbar, and particularly about 20 to 40 mbar or more particularly, about 30-35 mbar is used. The product may be dried after evaporation. In embodiments, isolation step (b) is initiated soon after the preparation of the mixture in step (a), for example within 1 hour, within 45 minutes, or within 30 minutes after preparation of the mixture in step (a). In embodiments, the isolation step (b) is carried out within 15 minutes, within 10 minutes or within 5 minutes after preparation of the mixture in step (a). Moreover, in embodiments, step (b) is conducted as rapidly as possible. In embodiments, step (b) comprises evaporation of the solvent at a temperature of about 40 to 60° C., or about 50° C., under vacuum (in embodiments, a reduced pressure of about 1 to about 100 mbar, about 10 to 50 mbar, and particularly about 20 to 40 mbar or more particularly, about 30 to 35 mbar is used). In embodiments, step (b) comprises removal of the solvent by evaporation within a time period of 1 hour, within 45 minutes, within 30 minutes, within 20 minutes, within 10 minutes or within 5 minutes.

In another embodiment of the process for preparing crystalline SAGE-217:oxalic acid, step (a) comprises providing a mixture containing SAGE-217 and oxalic acid in a slurry comprising cyclohexane or toluene, or a combination thereof, and optionally heating. Preferably the solvent is cyclohexane or toluene. The heating may be to a temperature of about 40 to about 80° C., about 45 to about 70° C., about 50 to about 65° C. The slurry may be stirred, preferably at the elevated temperature. The stirring is preferably carried out for a period of about 8 to about 96 hours, about 20 to about 65 hours, about 36 to about 55 hours or about 40 to about 50 hours.

In embodiments, the slurry in step (a) wherein cyclohexane or toluene is present at an amount of about 5 to 30 vol, about 10 to 20 vol, about 12 to 18 vol, about 14 to 16 vol, and preferably about 15 vol, in relation to SAGE-217.

In embodiments the slurry in step (a) has a mol ratio of SAGE-217 to oxalic acid of about 1:3 to about 1:1, about 1:2 to about 1:1, about 1:1.7 to about 1:1.3, or about 1 to about 1:5, about 1:1 to about 1:4. In other embodiments, the slurry in step (a) has a mol ratio of SAGE-217:oxalic acid of about 1:1 to about 1:0.5, or about 1:0.8 to about 1:0.5, or about 1:0.6 to about 1:0.5, or about 1:0.5. In other embodiments, the slurry in step (a) has a mol ratio of SAGE-217 to oxalic acid of about 1:0.8 to about 1:0.5, or about 1:0.6 to about 1:0.5, or about 1:0.5. In embodiments, the antisolvent in step (a) is selected from cyclohexane or toluene.

In embodiments, after stirring the slurry, the crystalline SAGE-217:oxalic acid may be isolated in step (b) by any suitable means such as by filtration or by centrifuge. In embodiments, the isolation is carried out by centrifuge. The product may be dried after isolating.

In another embodiment of the above process, the above crystalline SAGE-217:oxalic acid Form OCC1 can be prepared by a process including solvent/antisolvent crystallization. Thus, in an alternative embodiment of the above process, preparing a mixture comprising SAGE-217 and oxalic acid in one or more organic solvents according to step (a) comprises combining a solution of SAGE-217 and oxalic acid in solvent at room temperature, and adding an antisolvent. The process may further comprise the steps of: b) isolating crystalline SAGE-217:oxalic acid Form OCC1, optionally by filtration; and c) optionally drying. Preferably, the organic solvent is chloroform and the antisolvent is n-heptane. In embodiments, crystalline SAGE-217:oxalic acid form OCC1 is prepared by crystallization from a mixture of chloroform and n-heptane. In some embodiments, the process includes: a) combining a solution of SAGE-217 and oxalic acid in chloroform, preferably at room temperature, with heptane, preferably cooled heptane; b) optionally isolating crystalline SAGE-217:oxalic acid Form OCC1, optionally by filtration; and c) optionally drying.

In embodiments, step a) comprises dissolving SAGE-217 in chloroform at room temperature, optionally with stirring, optionally filtering the solution to remove insoluble material. The chloroform may be present at an amount of about 1 to about 20 vol, in embodiments about 5 to about 15 vol, and in other embodiments, about 10 vol of chloroform is used. In embodiments, about 0.5 to 1.0 equivalents of oxalic acid, preferably about 0.6 to 1.0 equivalents and preferably 0.6 equivalents of oxalic acid is added to the solution. The addition of the oxalic acid may form a slurry. Preferably, the mixture comprising SAGE-217 and oxalic acid in chloroform is at room temperature.

In embodiments, in step (a) heptane, is added to the mixture comprising SAGE-217 and oxalic acid in chloroform. The heptane may be added dropwise. The heptane is preferably cooled prior to combining. In embodiments, the heptane is cooled before addition to the mixture, preferably to a temperature of about −5° C. to about 15° C., about 1 to about 10° C., or preferably about 1 to about 5° C., or about 4° C. In embodiments, heptane is used in an amount of about 10 to 30 ml per gram of SAGE-217, about 20 to about 30 ml, and particularly about 22.5 ml per gram of SAGE-217. In embodiments, the mixture is stirred during the addition of heptane.

In embodiments, the ratio of chloroform to heptane is about 1:0.5 to about 1:5, about 1:1 to about 1:4, about 1:1.5 to about 1:3, or about 1:2 to about 1:2.5, or about 1:2.25.

Following the addition of heptane to SAGE-217 and oxalic acid solution, the mixture may be maintained, preferably with stirring, for a suitable period of time, in embodiments for about 1 to 48 hours, about 10 to about 30 hours, or about 23 hours. The mixture is preferably maintained at room temperature with stirring.

In any embodiment of the above process, step (b) may include isolation of crystalline SAGE-217:oxalic acid Form OCC1. The isolation may be done by filtering the suspension formed in step (a) optionally, under nitrogen environment. Alternatively the isolation can be carried out by centrifuge. Following the isolation, the product may be washed, and optionally dried.

In any embodiment of the above described processes, the product may be dried after isolating. In embodiments, the drying is carried out in a vacuum oven. The drying step (c) may be carried out at any suitable temperature, for example, at about 45 to about 70° C., about 45 to about 55° C. or about 55° C. in a vacuum oven. Any suitable drying time may be used, for example, in embodiments the drying is carried out over a period of about 6 to about 120 hours, about 10 to about 24 hours, about 12 to about 20 hours, or about 16 hours. The drying may be conducted at reduced pressure or a vacuum. When the drying is carried out under reduced pressure or a vacuum, a reduced pressure of about 1 or about 200 mbar, about 1 to about 100 mbar, about 1 to 50 mbar, and particularly about 5 to about 40 mbar or more particularly, about 20 mbar, is used. Alternatively, the drying step (c) may be carried out under a flow of nitrogen or air or under vacuum. Drying under a flow of nitrogen or air may be performed at a temperature of about 20 to about 50° C., about 40 to about 50° C., and in some embodiments about 45° C.

In any embodiment of the above described processes, the crystalline SAGE-217: oxalic acid may be further purified, for example to remove any traces of oxalic acid impurity. The purification process in embodiments may comprise washing the crystalline SAGE-217: oxalic acid in a solvent which has a greater selectively to dissolve oxalic acid in preference to SAGE-217:oxalic acid. In any embodiment, the purification process comprises: (i) stirring a slurry comprising crystalline SAGE-217:oxalic acid in a suitable solvent, and (ii) isolating the purified crystalline SAGE-217:oxalic acid. The suitable solvent can be one solvent, or a combination of two or more solvents which selectively dissolves oxalic acid and in which crystalline SAGE-217:oxalic acid is not soluble or at least does not dissolve in an appreciable amount. Suitable solvents include esters. In embodiments, the solvent can be a C₄-C₁₀ ester, a C₅-C₈ ester, a C₅-C₆ ester, or isopropyl acetate. The slurrying can be carried out at any suitable temperature in which the oxalic acid can dissolve in the selected solvent, and wherein the crystalline SAGE-217:oxalic acid is not soluble, or at least does not dissolve in any appreciable amount. The crystalline SAGE-217:oxalic acid and solvent may be mixed to form a slurry, and the slurry may be stirred. Typically, the slurry can be stirred at room temperature. The slurry can be stirred for a sufficient time to enable the oxalic acid to dissolve in the solvent. In embodiments, the mixture can be stirred for: about 2 to about 24 hours, about 4 to about 16 hours, about 5 to about 10 hours, about 6 to about 8 hours, or about 7.5 hours. The crystalline SAGE-217:oxalic acid may be isolated from the mixture by any suitable method such as by filtration or by centrifuge, preferably by centrifuge.

The purified crystalline SAGE-217:oxalic acid may be dried, preferably in a vacuum oven. The drying may be carried out at a temperature of about 45° C. to about 70° C., about 45° C. to about 55° C., or about 50° C. in a vacuum oven. The drying is conducted for a sufficient time to remove the solvent(s). In embodiments, the drying is carried out over a period of about 1 to about 40 days, about 10 to about 30 days, about 15 to about 25 days, or about 20 days. The drying may be carried out at reduced pressure, in embodiments, at a pressure of about 1 or about 200 mbar, about 1 to about 100 mbar, about 1 to 50 mbar, and more preferably about 5 to about 40 mbar, or more particularly, about 20 mbar.

The above processes for preparing crystalline SAGE-217:oxalic acid Form OCC1 according to any of the described embodiments may further include step of combining the crystalline SAGE-217:oxalic acid Form OCC1 with at least one pharmaceutically acceptable excipient to form a pharmaceutical composition of pharmaceutical combination.

The present disclosure provides the above described crystalline SAGE-217:oxalic acid for use in the preparation of pharmaceutical compositions including SAGE-217:oxalic acid and/or crystalline polymorphs thereof.

The present disclosure also encompasses the use of crystalline SAGE-217:oxalic acid of the present disclosure for the preparation of pharmaceutical compositions of SAGE-217: oxalic acid and/or crystalline polymorphs thereof.

The present disclosure includes processes for preparing the above mentioned pharmaceutical compositions. The processes include combining crystalline SAGE-217:oxalic acid of the present disclosure with at least one pharmaceutically acceptable excipient.

Pharmaceutical formulations of the present invention contain crystalline SAGE-217: oxalic acid of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, crystalline SAGE-217:oxalic acid and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention can be a capsule containing the composition, in embodiments a powdered or granulated solid composition of the present disclosure, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

A pharmaceutical formulation of crystalline SAGE-217:oxalic acid can be administered. Crystalline SAGE-217:oxalic acid may be formulated for administration to a mammal, in embodiments a human, by injection. Crystalline SAGE-217:oxalic acid can be formulated, for example, as a viscous liquid solution or suspension, in embodiments a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. See, e.g., Ansel et al., Pharmaceutical Dosage Forms and Drug Delivery Systems, 7th ed.

The crystalline polymorph of SAGE-217:oxalic acid of the present disclosure and their pharmaceutical compositions and/or formulations can be used as medicaments, preferably for treatment of patients with depression disorder, major depressive disorder, postpartum depression, insomnia, bipolar depression, essential tremor, Parkinson's disease and/or dyskinesias. In embodiments, the crystalline polymorph of SAGE-217:oxalic acid of the present disclosure and their pharmaceutical compositions and/or formulations can be used as medicaments for treatment of patients with major depressive disorder or postpartum depression.

The present disclosure also provides methods of treating depression by administering a therapeutically effective amount of crystalline SAGE-217:oxalic acid of the present disclosure, or at least one of the above pharmaceutical compositions and/or formulations, to a subject in need of the treatment.

Having thus described the disclosure with reference to exemplary embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to, limit its scope in any way.

### Powder X-Ray Diffraction (“XRPD”) Method

The sample was powdered in a mortar and pestle and applied directly on a silicon plate holder. The X-ray powder diffraction pattern was performed on X-Ray powder diffractometer. Powder X-ray Diffraction was performed on ARL (SCINTAG) powder X-Ray diffractometer model X'TRA equipped with a solid state detector. Copper radiation of 1.5418 Å was used. Scanning parameters: range: 2-40 degrees two-theta; scan mode: continuous scan; step size: 0.05°, and a rate of 3 deg/min.

### Preparation of Starting Materials

SAGE-217 used for the below examples can be prepared according to any procedure known from the literature. SAGE-217 Form A used in example below can be prepared according to PCT Patent Application Publication No. WO 2018/039378.

### Example 1. Preparation of Crystalline SAGE-217: Oxalic Acid Form OCC1

Methanol (1.2 ml, 40V) was added to SAGE-217 (50 mg, 0.12 mmol) and oxalic acid (14 mg, 0.16 mmol, 1.3 eq) to give slurry. The obtained slurry was heated to 60° C. to obtain complete dissolution followed by mechanically filtration using filter disk. The obtained clear mother-liquor was evaporated upon 50° C./300-35 mbar to give a solid. The obtained solid was dried in vacuum oven at 45° C. for 7 hours to afford white solid, which was characterized by X-ray powder diffraction a solid as crystalline SAGE-217:oxalic acid Form OCC1 (FIG. 2).

### Example 2. Preparation of Crystalline SAGE-217: Oxalic Acid Form OCC1

SAGE-217 (Form A, 30 mg, 0.07 mmol) and oxalic acid (13 mg, 0.15 mmol, 2 eq) was grinded with 2 drops of MeOH using mortar and pestle for 1 minute at room temperature. The obtained solid was characterized by X-ray powder diffraction as crystalline SAGE-217:oxalic acid Form OCC1.

### Example 3. Preparation of Crystalline SAGE-217: Oxalic Acid Form OCC1

Cyclohexane or Toluene (7.5 ml, 15V) and oxalic acid (165 mg, 1.8 mmol, 1.5 eq) were added to SAGE-217 (Form A, 500 mg, 1.2 mmol) to obtain a slurry. The slurry was magnetically stirred at 60° C. over a period of 48 hours. The solid was then filtered by centrifuge to afford a white wet solid. The obtained solid was dried in vacuum oven at 50° C. for 16 hours to afford white solid, which was characterized by X-ray powder diffraction as SAGE-217:oxalic acid crystal Form OCC1.

### Example 4. Preparation of Crystalline SAGE-217: Oxalic Acid Form OCC1

Chloroform (10 ml, 10V) was added to SAGE-217 (1 gram, 2.4 mmol) to obtain a slurry. The slurry was magnetically stirred at RT over a period of 30 minutes to obtain complete dissolution follows by mechanically filtration using filter disk. Next, an oxalic acid (132 mg, 1.4 mmol, 0.6 eq) was added to give light slurry. Then, the cold heptane as anti-solvent (22.5 ml) was added drop-wise to the stirred light slurry at RT and massive precipitation occurs. The obtained solid was stirred at room temperature over a period of 23 hours. The obtained solid was then filtered by centrifuge and dried in vacuum oven at 50° C. over a period of 3 days to afford white solid, which was characterized by X-ray powder diffraction as SAGE-217:oxalic acid crystal Form OCC1.

### Example 5. Preparation of Crystalline SAGE-217: Oxalic Acid Form OCC1

Methanol (1.2 ml, 24V) was added to SAGE-217 (50 mg, 0.12 mmol) and oxalic acid (14 mg, 0.16 mmol, 1.3 eq) to give a slurry. The obtained slurry was heated to 60° C. to obtain complete dissolution followed by mechanically filtration using filter disk. The obtained clear mother-liquor was evaporated upon 50° C./30-35 mbar to give a solid. The obtained solid was dried in vacuum oven at 45° C. for 7 hours to afford white solid, which was characterized by X-ray powder diffraction a solid as crystalline SAGE-217:oxalic acid Form OCC1.

### Example 6. Purification of Crystalline SAGE-217: Oxalic Acid Form OCC1

Isopropyl acetate (5 ml, 10V) was added to sample containing crystalline SAGE-217: Oxalic acid Form OCC1 (500 mg, 1.2 mmol), prepared according to Example 4 (following Example 4 but only using 0.8 eq of oxalic acid), to obtain a slurry. The slurry was magnetically stirred at RT over a period of about 7.5 hours. The solid was then filtered by centrifuge and dried in vacuum oven at 50° C. over a period of 20 days to afford a white solid, which was characterized as crystalline SAGE-217: Oxalic acid crystal Form OCC1 without residue of oxalic acid.

1. Crystalline SAGE-217:oxalic acid.

2. Crystalline SAGE-217:oxalic acid which is a co-crystal.

3. Crystalline SAGE-217:oxalic acid according to claim 2, wherein the molar ratio between SAGE-217 and oxalic acid is between: 2:1.5 and 1.5.

4. A crystalline product according to claim 1, designated form OCC1, which is characterized by data selected from one or more of the following:
a. an XRPD pattern having peaks at 6.2, 12.5, 14.0, 15.2 and 18.9 degrees 2-theta±0.2 degrees 2-theta;
b. an XRPD pattern as depicted in FIG. 2; or
c. combinations of these data.

5. A crystalline product according to claim 1, designated form OCC1, characterized by an XRPD pattern having peaks at 6.2, 12.5, 14.0, 15.2 and 18.9 degrees 2-theta±0.2 degrees 2-theta, and also having one, two, three, four or five additional peaks selected from 10.8, 16.5, 17.9, 18.5 and 20.7 degrees two theta±0.2 degrees two theta.

6. A crystalline product according to claim 1, designated form OCC1, characterized by an XRPD pattern having peaks at 6.2, 10.8, 12.5, 14.0, 15.2, 16.5, 17.9, 18.5, 18.9, and 20.7 degrees 2-theta±0.2 degrees 2-theta.

7. A crystalline product according to claim 1, designated form OCC1 wherein the crystalline form is anhydrous form.

8. A crystalline product according to claim 1, designated form OCC1, which contains: no more than about 20%, of any other crystalline forms of SAGE-217:oxalic acid.

9. A crystalline product according to claim 1, designated form OCC1, which contains: no more than about 20% of amorphous SAGE-217:oxalic acid.

10-52. (canceled)

53. A process according to claim 1, further comprising a step of combining the crystalline SAGE-217:oxalic acid Form OCC1 with at least one pharmaceutically acceptable excipient to form a pharmaceutical composition.

54. (canceled)

55. A pharmaceutical composition comprising a crystalline product according to claim 1, and at least one pharmaceutically acceptable excipient.

56. (canceled)

57. (canceled)

58. A process for preparing a pharmaceutical composition comprising combining the crystalline product according to claim 1 with at least one pharmaceutically acceptable excipient.

59. A crystalline product according to claim 1, for use as a medicament.

60. A crystalline product according to claim 1 for use in the treatment of a CNS related disorder.

61. A method of treating a CNS related disorder selected from postpartum depression (PPD), major depressive disorder (MDD), bipolar depression, insomnia essential tremor (ET), Parkinson's disease, and dyskinesias, comprising administering a therapeutically effective amount of a crystalline product according to claim 1 to a subject in need of the treatment.
